(19) United States
(12) Patent Application Publication
(10) Pub. No.: US 2010/0241070 A1
(43) Pub. Date: Sep. 23, 2010

(54) BALLOON CATHETER FOR DISTAL PROTECTION COMPATABILITY

(75) Inventors: John Blix, Maple Grove, MN (US); William R. Little, Medway, MA (US); Jamie Lockwood, Bridgewater, MA (US)

Correspondence Address: CROMPTON, SEAGER & TUFTE, LLC, 1221 NICOLLET AVENUE, SUITE 800, MINNEAPOLIS, MN, 55403-2420, US

(73) Assignee: BOSTON SCIENTIFIC SCIMED, INC., Maple Grove, MN (US)

(21) Appl. No.: 12/792,195

(22) Filed: Jun. 2, 2010

Related U.S. Application Data
(63) Continuation of application No. 11/271,653, filed on Nov. 10, 2005.

(51) Int. Cl. A61M 25/10

(52) U.S. Cl. 604/96.01

(57) ABSTRACT

A balloon catheter that may include a catheter shaft having a proximal end and a distal end, the catheter shaft having a core having an outer surface, the core extending to the distal end of the catheter shaft, the catheter shaft having a tubular member disposed about the core, the tubular member having an inner surface and an outer surface, and an inflation lumen between the tubular member inner surface and the tubular member outer surface, and an inflatable member having a proximal cone, a distal cone and a central section therebetween, the central section having proximal and distal ends, the proximal and distal cones defining an inflation cavity, the inflatable member further comprising a proximal waist affixing the proximal cone to the outer surface of the tubular member and a distal waist affixing the distal cone to the outer surface of the core, wherein the inflatable member is configured such that the distance along the longitudinal axis between the distal end of the central section and the distal waist is less than the distance between the proximal end of the central section and the proximal waist, wherein the inflation lumen is fluidly connected to the inflation cavity.

### RELATED APPLICATIONS

This application is a continuation of U.S. application Ser. No. 11/271,653 filed Nov. 10, 2005.

### FIELD

The present invention relates generally to the field of medical devices having an expandable balloon disposed proximate a distal portion of a shaft, such as angioplasty balloon catheters for example. More specifically, the present invention relates to configurations for improved compatibility with distal protection devices.

### BACKGROUND

Intravascular diseases are commonly treated by relatively non-invasive techniques such as percutaneous transluminal angioplasty and percutaneous transluminal coronary angioplasty. These therapeutic techniques are well known in the art and typically involve the use of a balloon catheter with a guidewire, possible in combination with other intravascular devices such as stents. A typical balloon catheter has an elongate shaft with a balloon attached proximate the distal end and a manifold attached to the proximal end. In use, the balloon catheter is positioned adjacent a restriction in a diseased vessel. The balloon is then inflated and the restriction in the vessel is opened. Sometimes a distal protection device, such an embolic filter, is used. The distal protection device may be on a distal part of the guidewire. In some vascular vessels, such as certain carotids and renal vessels, space is extremely limited. An adequate amount of space between the distal protection device and the balloon is desired. There is thus an ongoing need to provide alternative balloon catheter configurations that can be used in confined vessels with distal protection devices.

### SUMMARY

The invention provides such balloon catheter alternatives to provide these and other improvements.

One embodiment pertains to a balloon catheter that may comprise a catheter shaft having a proximal end and a distal end, the catheter shaft having a core having an outer surface, the core extending to the distal end of the catheter shaft, the catheter shaft having a tubular member disposed about the core, the tubular member having an inner surface and an outer surface, and an inflation lumen between the tubular member inner surface and the tubular member outer surface, and an inflatable member that may have a proximal cone, a distal cone and a central section therebetween, the central section having proximal and distal ends, the proximal and distal cones defining an inflation cavity, the inflatable member further comprising a proximal waist affixing the proximal cone to the outer surface of the tubular member and a distal waist affixing the distal cone to the outer surface of the core, wherein the inflatable member may be configured such that the distance along the longitudinal axis between the distal end of the central section and the distal waist is less than the distance between the proximal end of the central section and the proximal waist, wherein the inflation lumen is fluidly connected to the inflation cavity. The distal waist may extend proximally from the distal cone to provide a distal balloon portion having an inverted attachment. In another embodiment, the distal cone has a first section that tapers distally from the central section and a second section that tapers proximally to the distal waist. In another embodiment, the distal cone tapers proximally to the distal waist. The inflatable member may comprise a compliant balloon material, a non-compliant polymeric material, a reinforced polymeric material or other suitable material. The balloon waits may be attached to the catheter shaft with an adhesive bond, a thermal bond or other suitable bond. The catheter may have a stent or other medical device mounted thereon. The core outer surface and the tubular member inner surface may define the inflation lumen, and the core may comprise a guidewire lumen extending to an opening at the distal end of the catheter shaft. The catheter shaft distal end may terminate distal the inflatable member distal end.

Another embodiment pertains to a balloon catheter that may comprise a catheter shaft having an outer surface, an inflation lumen and a guidewire lumen, and a balloon having a balloon wall, a proximal cone, a distal cone and a cylindrical central section therebetween, the balloon affixed to the catheter shaft with a proximal waist extending distally from the proximal cone and a distal waist extending proximally from the distal cone, the balloon proximal and distal cones and central section defining an inflation lumen in contact with a first surface of the balloon wall, the balloon wall having a second surface opposite the first surface. The proximal cone may taper proximally from the central section and the distal cone may taper distally from the central section. The balloon wall second surface may face the catheter shaft outer surface at the balloon proximal and distal waists.

Another embodiment pertains to a method of making a balloon catheter that may include the steps of forming a balloon having a proximal waist, a proximal cone, a central cylindrical section, a distal cone and a distal waist, providing a catheter having an outer surface and a distal end, everting the distal waist such that it extends proximally from the distal cone, sliding the balloon onto the catheter, and bonding the balloon proximal and distal waists to the catheter. The method may further comprise the steps of providing an adhesive, and disposing the adhesive on the catheter outer surface, and the steps of sliding the balloon over the catheter distal end onto the catheter proximally past the location of the distal waist bond and subsequently applying the adhesive, and sliding the balloon distally to the adhesive to form the bond between the distal waist and the catheter outer surface.

The above summary of the present invention is not intended to describe each disclosed embodiment or every implementation of the present invention. The Figures, Detailed Description and Examples which follow more particularly exemplify these embodiments.

### BRIEF DESCRIPTION OF DRAWINGS

The invention may be considered more completely understood in consideration of the following detailed description of various embodiments of the invention in connection with the accompanying drawings in which:

FIG. 1 depicts a plan view of a balloon catheter disposed on a distal protection guidewire;

FIG. 2 is a diagrammatic cross-sectional view of a distal portion of a balloon catheter;

FIG. 2A is a diagrammatic cross-sectional view of a variation on the balloon catheter of FIG. 2.

FIG. 3 is a diagrammatic cross-sectional view of a distal portion of another balloon catheter;

FIG. 4 is a diagrammatic cross-sectional view of a distal portion of another balloon catheter;

FIG. 5 is a diagrammatic cross-sectional view of a distal portion of another balloon catheter;

FIG. 6 is a diagrammatic cross-sectional view of a distal portion of another balloon catheter; and

FIG. 7 is a diagrammatic cross-sectional view of a distal portion of another balloon catheter.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

The following description should be read with reference to the drawings wherein like reference numerals indicate like elements throughout the several views. The drawings, which are not necessarily to scale, depict illustrative embodiments of the claimed invention.

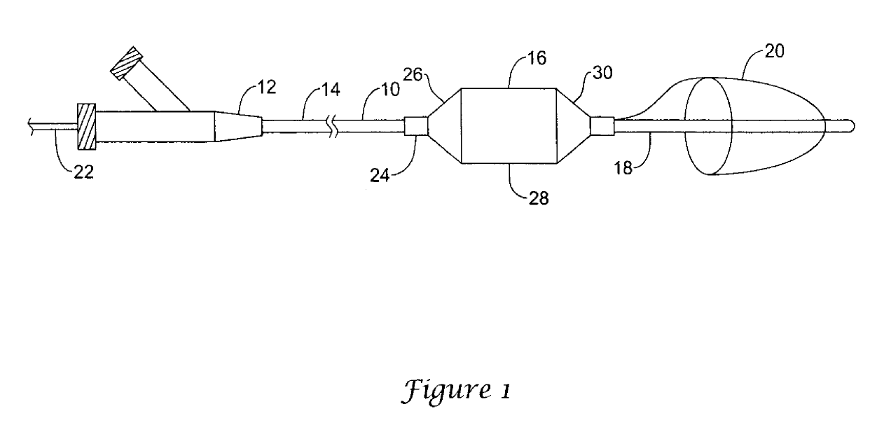

A balloon catheter 10 having a hub and strain relief assembly 12 having two luer-type valves, an elongate catheter shaft 14 and an inflatable balloon 16 is disposed on a distal protection guidewire 18 having a distal protection device 20 disposed on a wire 22, as shown in FIG. 1. There are many means for connecting a balloon to a catheter shaft and the means illustrated are not meant to be limiting, but merely illustrative. The balloon includes a proximal waist 24, a proximal cone 26, a central section 28 and a distal cone 30. It also includes a distal waist 32 (not pictured). These sections of the balloon are part of one continuous balloon and are distinguished only to illustrate the features of the balloon. As is the case in many conventional balloon catheters, the proximal waist 24 extends proximally from the proximal cone 26 and is bonded to catheter shaft 14. Unlike the proximal waist and as will be illustrated in more detail below, distal waist 32 is everted and extends proximally from distal cone 30 and is bonded to catheter shaft 14. As can be seen, this arrangement shortens the balloon 16 distal of the central section 28 to create a shortened landing zone, which permits the central section of the balloon to be positioned closer to the distal protection device 20.

Balloon catheter 10 may be an angioplasty catheter, a stent delivery catheter, a cutting balloon catheter, or other types of balloon catheters. The hub and strain relief assembly 12 shown is merely illustrative. Balloon catheter 10 may include any suitable hub or hub and strain relief or have any suitable proximal terminal assembly. The catheter shaft 14 includes an inflation lumen fluidly connected to the balloon 16 and a guidewire lumen in which distal protection guidewire 18 is disposed. The catheter shaft may have other lumens if desired. For example, it may have a lumen for the delivery of therapeutic agents or a second inflation lumen for a second balloon. The catheter shaft is depicted as a coaxial configuration; other configurations are possible. The various tubular members that make up catheter shaft 14 are fixed with respect to each other. For example, they may be fixed towards the distal end to increase columnar support. Further, the catheter shaft and hub may be any suitable configuration. For example, in some embodiments a single-operator-exchange configuration, where the guidewire lumen extends along only a distal portion of the catheter shaft, is desirable. The balloon catheter is depicted as disposed on a distal protection guidewire, but it is not so limited. The balloon catheter may be disposed on any suitable device. For example, it may be disposed on a guidewire, an atherectomy device, a balloon catheter, a distal protection guidewire having a different distal protection device or other suitable apparatus.

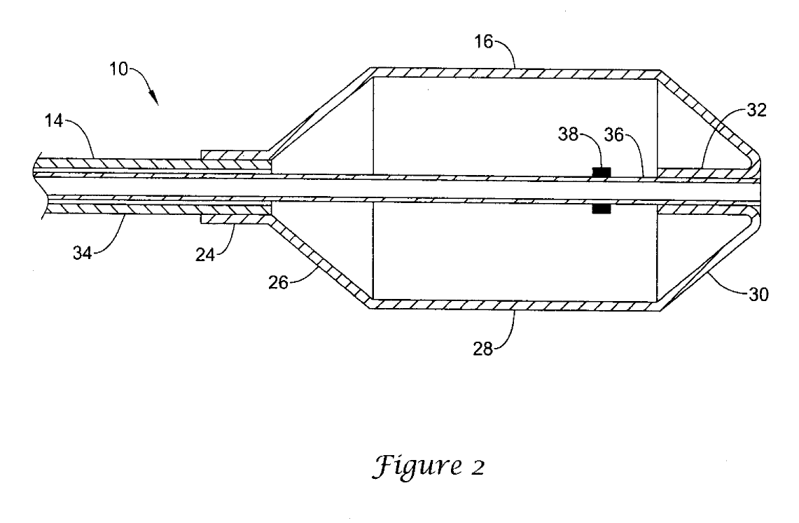

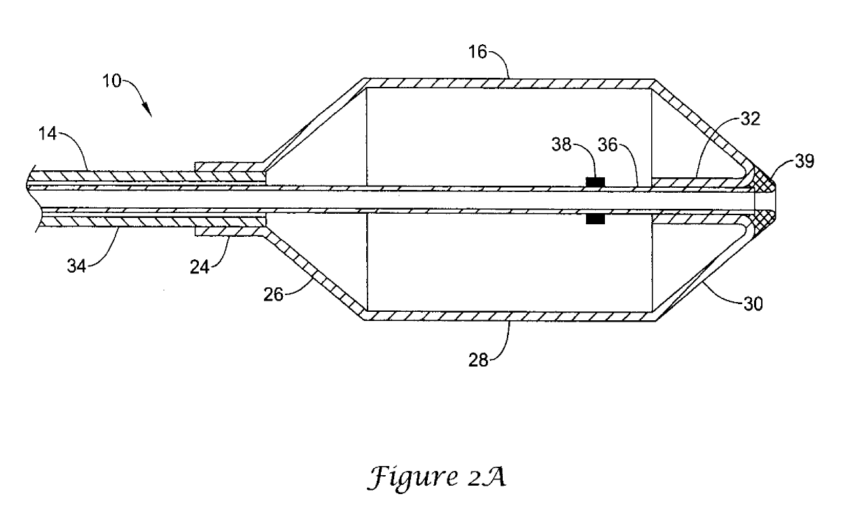

Turning now to FIG. 2, which is a diagrammatic cross-sectional view of a distal portion of balloon catheter 10, distal waist 32 is illustrated. Catheter shaft 14 has outer tubular member 34 and inner tubular member 36. An example radiopaque marking, radiopaque marker band 38, which is shown disposed on inner tubular member 36, may be disposed in a different location or on a different component. Catheter shaft 14 is shown in a coaxial configuration, though other configurations are possible. For example, configurations with an off-center inner tubular member or having side-by-side tubular members are contemplated. Central balloon section 28 is selected to have a desired size and proximal cone 26 tapers proximally therefrom to proximal waist 24. Distal waist tapers distally from the distal end of the central section to a distal waist 32. As illustrated, distal waist 32 extends proximally from the distal cone. The balloon may be formed in this configuration or the balloon may be formed with distal waist 32 extending distally from the distal cone and then the distal waist is everted to the configuration shown. The distal waist may be bonded to inner tubular member 36 at the distal end thereof or may be bonded at a location proximal to the distal end. FIG. 2A is a diagrammatic cross-sectional view of a catheter 10 having a tapering distal tip 39, which nevertheless retains a shortened landing zone over embodiments that have a distal waist extending distally from the distal cone.

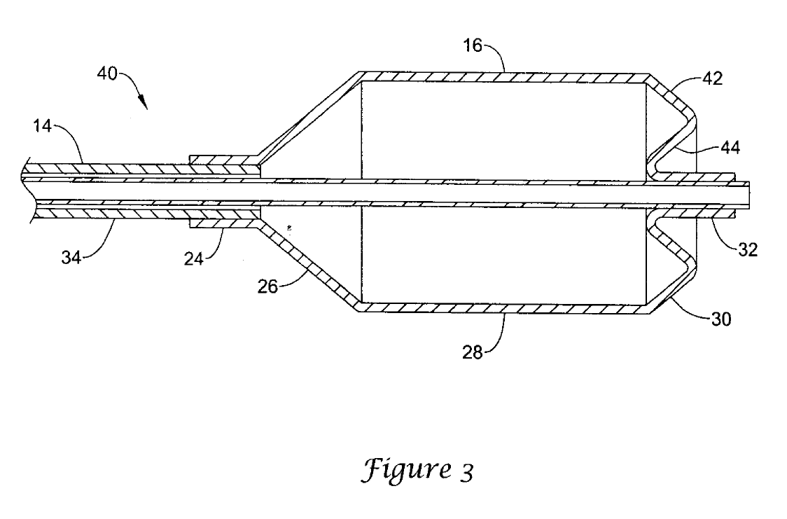

FIG. 3 is a diagrammatic cross-sectional view illustrating the distal portion of a balloon catheter 40. Balloon 16 is disposed on a catheter shaft 14. Catheter shaft 14 has an inner tubular member 36 defining a guidewire lumen and an outer tubular member 34 which together with the inner tubular member defines the inflation lumen. While catheter shaft 14 is depicted as extending slightly beyond the distal waist, the catheter shaft 14 could terminate distally at the distal waist, further shortening the distal landing zone. Balloon 16 includes proximal waist 24, proximal cone 26, central section 28, distal cone 30 and distal waist 32. Proximal waist 24 extends proximally from central section 28. Distal cone 30 has a first portion 42 tapering from the central section distally and second portion 44 tapering from the first portion proximally. The distal waist 32 extends distally from the distal cone 30. As is illustrated in the figure, this distal cone configuration shortens the length of the distal cone, shortening the length of the distal landing zone. Of course, variations are possible. For example in one embodiment, distal cone 32 is everted and consequently extends proximally from the distal cone. Likewise, the proportions of the first portion and the second portion may be varied with respect to each other.

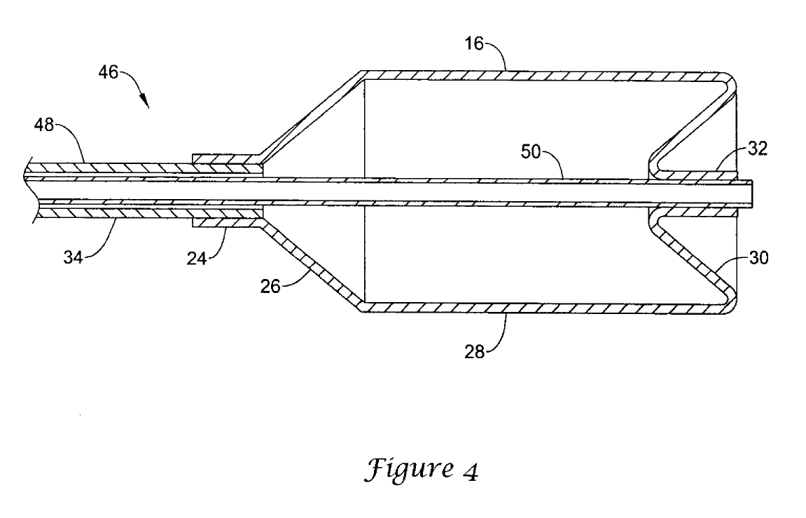

FIG. 4 is a diagrammatic cross-sectional view illustrating the distal portion of a balloon catheter 46, which is similar to balloon catheter 40 except for the variations discussed below. The distal cone 30 of the balloon tapers proximally from the distal end of balloon central section 28 to distal waist 32, which extends distally from the distal cone 30. Balloon 16 is mounted on a catheter shaft 48 that has an outer tubular member and an off-center inner tubular member 50. In some configurations, catheter shaft 48 can terminate at the distal end of distal waist 32, further shortening the landing zone.

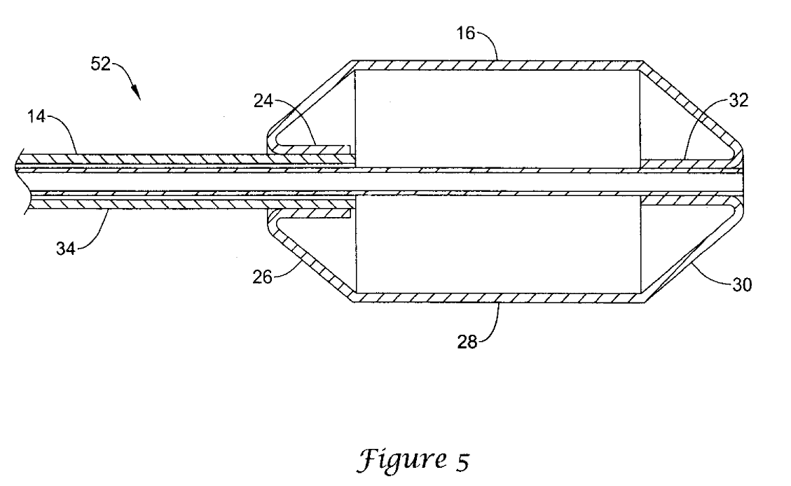

FIG. 5 is a diagrammatic cross-sectional view illustrating the distal portion of a balloon catheter 52. Balloon 16, mounted on catheter shaft 14, includes proximal waist 24 bonded to catheter shaft 14, proximal cone 26, central section 28, distal cone 30 and distal waist 32 bonded to catheter shaft 14. Proximal and distal cones 26 and 30 extend proximally and distally, respectively, from balloon central section 26. Proximal waist 24 extends distally from proximal cone 26 and distal waist 32 extends proximally from distal waist 30. This creates a more symmetric configuration that has a shortened distal landing zone.

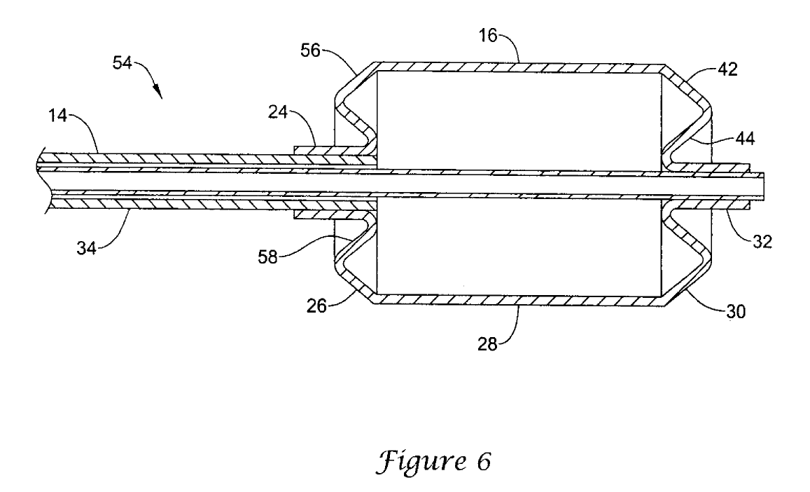

FIG. 6 is a diagrammatic cross-sectional view illustrating the distal portion of a balloon catheter 54. Balloon 16, mounted on catheter shaft 14, includes proximal waist 24 bonded to catheter shaft 14, proximal cone 26, central section 28, distal cone 30 and distal waist 32 bonded to catheter shaft 14. Distal cone 30 includes a first portion 42 tapering distally from balloon center section 26 and a section portion 44 tapering proximally from first portion 42 to distal waist 32. Proximal cone 30 includes a first portion 56 tapering proximally from balloon center section 16 and a section portion 58 tapering distally from first portion 56 to proximal waist 24. Proximal waist 24 extends proximally from proximal cone 26 and distal waist 32 extends distally from distal waist 30. In other contemplated embodiments, proximal waist 24 may extend distally from proximal cone 26 and distal waist 30 may extend proximally from distal cone 30.

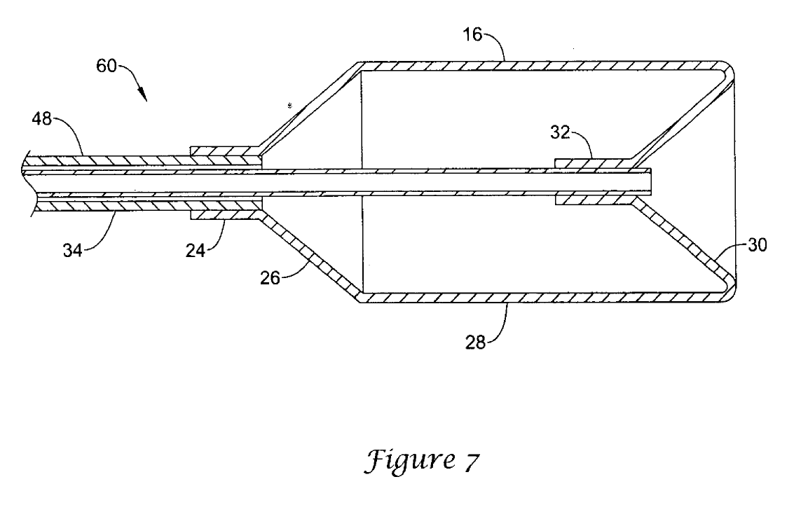

FIG. 7 is a diagrammatic cross-sectional view illustrating the distal portion of a balloon catheter 60, in which a balloon 16 is mounted on a catheter shaft 14. The balloon has a central section 28 from which both proximal and distal cones 26 and 30 taper in a proximal direction. Proximal waist 24 extends proximally from proximal cone 26 and distal waist 32 extends proximally from distal cone 30. The distal end of catheter shaft 14 may terminate at the distal end of the distal waist, where the distal waist joins the distal cone to further shorten the landing zone.

Such a balloon catheter may be made according to the following method. An inflatable balloon having a distally extending distal cone and waist may be molded in a conventional manner. This balloon is then molded in a secondary process in which the distal cone and waist are everted and placed in a mold having a reverse-cone shape. The balloon is then low-pressure inflated over a mandrel and exposed to a temperature sufficient to cause a permanent set to keep the cone reverse after molding. Alternatively, the distal cone and waist can be everted and bonded to the catheter shaft with no heat set. The balloon may be slid proximally onto the distal end of the catheter shaft proximally past the distal waist bonding location. An adhesive or epoxy may be applied to the catheter shaft and the balloon may be slid back into position to bond the distal waist in place. In another variation a section of heat shrink tubing is placed over the everted distal waist. When heat is applied, the tubing applies pressure to the distal waist. The proximal waist may be bonded using conventional means. In another method, the balloon is placed on the catheter and the proximal waist is bonded using conventional means. A tool is used to hold the everted distal cone in place and the balloon is low pressure inflated. A mandrel is slid into the guidewire lumen to apply heat at the location of the distal waist to create a thermal bond between the catheter shaft and the distal waist.

The catheter in any of the embodiments may be made from any suitable materials and using any suitable construction. For example, the catheter may include a metallic hypotube section made from stainless steel or a nickel-titanium alloy. It may include a polymeric layer and may have a reinforcing layer such as a braid embedded therein. It may have section of varying rigidity and may have inner or outer liners having a low coefficient of friction such as a polytetrafluoroethylene (PTFE) liner. The polymer may be selected based on desired criteria such as strength and flexibility. Suitable polymers may include thermoplastics, polyesters, polyurethanes, elastomeric polyamides, and silicones.

The balloon material in any of the embodiments varies depending on the compliance of the balloon material desired. In general, the balloon material desired for the embodiments is either a polyether block amide (PEBAX), or polyethylene. When a compliant balloon material is desired, low pressure, relatively soft or flexible polymeric materials such as thermoplastic polymers, thermoplastic elastomers, polyethylene (high density, low density, intermediate density, linear low density), various co-polymers and blends of polyethylene, ionomers, polyesters, polyurethanes, polycarbonates, polyamides, poly-vinyl chloride, acrylonitrile-butadiene-styrene copolymers, polyether-polyester copolymers, and polyetherpolyamide copolymers are preferred. When a non-compliant balloon material is desired, materials having relatively rigid properties such as poly(ethylene terphthalate), polyimide, thermoplastic polyimide, polyamides, polyesters, polycarbonates, polyphenylene sulfides, polypropylene and rigid polyurethanes are desired.

It should be understood that this disclosure is, in many respects, only illustrative. Changes may be made in details, particularly in matters of shape, size, and arrangement of steps without exceeding the scope of the invention. The invention's scope is, of course, defined in the language in which the appended claims are expressed.

1. A method of deploying a therapeutic balloon catheter intravascularly, comprising the steps of:
obtaining an elongate member having a proximal end, a distal end, and a distal protection device proximate the distal end, the distal protection device having a compact configuration and a deployed configuration capable of capturing emboli;
advancing the distal protection device in the compact configuration to a region of interest in a vascular system;
at the region of interest, deploying the distal protection device to the deployed configuration;
obtaining a balloon catheter having a distal end, a proximal end, a balloon, a through lumen extending from the distal end to an opening proximal the balloon, and an inflation lumen fluidly connected to the balloon, the balloon having a proximal waist, a proximal cone, a central section, a distal waist and a distal cone disposed along a longitudinal axis, the distal waist and distal cone defining a shortened landing zone;
advancing the balloon catheter over the elongate member to the region of interest;
positioning the shortened landing zone adjacent the distal protection device; and
inflating the balloon.

2. The method of claim 1 wherein the distal end of the balloon catheter terminates at the distal waist.

3. The method of claim 1 wherein the distal cone has a portion that tapers proximally.

4. The method of claim 1 wherein the distal waist is everted.

5. The method of claim 1 wherein the location of interest is in a carotid artery.

6. The method of claim 1 wherein the location of interest is in a renal artery.

7. The method of claim 1, wherein a distal end of the distal waist is directly affixed to the distal end of the balloon catheter.

8. The method of claim 1, wherein the balloon is configured such that the distance along the longitudinal axis between a distal end of the central section and a proximal end of the distal waist is less than the distance between a proximal end of the central section and a distal end of the proximal waist.

9. The method of claim 1, wherein the distal waist extends proximally from the distal cone.

10. The method of claim 1, wherein the distal cone has a first section that tapers distally from the central section and a second section that tapers proximally to the distal waist.

11. The method of claim 1, wherein the balloon comprises a compliant balloon material.

12. The method of claim 1, wherein the balloon comprises a non-compliant polymeric material.

13. The method of claim 1, wherein the balloon comprises a reinforced polymeric material.

14. The method of claim 1, wherein the distal waist is attached to the balloon catheter with an adhesive bond.

15. The method of claim 1, wherein the distal waist is attached to the balloon catheter with a thermal bond.

16. The method of claim 1, wherein the balloon catheter further comprises a stent mounted on the central section of the balloon.

17. The method of claim 1, wherein the balloon catheter comprises an inner tubular member and an outer tubular member.

18. The method of claim 1, wherein an exterior surface of the balloon faces an outer surface of the balloon catheter at the proximal waist and an outer surface of the elongate member at the distal waist.

19. The method of claim 1, wherein the step of positioning the shortened landing zone adjacent the distal protection device further comprises positioning at least a portion of the distal protection device proximal of a distalmost extent of the balloon.

20. The method of claim 17, wherein the inner tubular member and the outer tubular member are fixed with respect to each other.
